(19) United States
(12) Patent Application Publication
(10) Pub. No.: US 2018/0028424 A1
(43) Pub. Date: Feb. 1, 2018

(54) COMPOSITIONS FOR IMPROVING THE HEALTH OF HAIR, SKIN, AND NAILS

(71) Applicant: FYBRANDS CORP., Salt Lake City, UT (US)

(72) Inventors: James Humphreys, Victoria (CA); Max Sabour, Salt Lake City, UT (US)

(21) Appl. No.: 15/553,740

(22) PCT Filed: Feb. 25, 2016
(86) PCT No.: PCT/US16/19624

§ 371 Date: Aug. 25, 2017

Related U.S. Application Data
(60) Provisional application No. 62/120,704, filed on Feb. 25, 2015.

(51) Int. Cl. A61K 8/49; A61K 8/92; A61Q 5/00; A61Q 19/00; A61Q 3/00; A61K 8/37; A61Q 7/00

(52) U.S. Cl. CPC A61K 8/498; A61K 8/37; A61K 8/922; A61Q 7/00; A61Q 19/00; A61Q 3/00; A61Q 5/00; A61K 2800/48

(57) ABSTRACT

Compositions for improving the health of human hair, skin, and nails, and methods of using and preparing such compositions are disclosed.

### TECHNICAL FIELD

The present disclosure is directed to compositions for improving the health of human hair, skin, and nails, e.g., by stimulating hair growth, and methods of preparing such compositions.

### BACKGROUND

Most men and women want healthy, beautiful hair, skin, and nails. Moreover, these tissues serve important physiological functions. Skin, for example, forms a protective barrier against infection by pathogens. Unfortunately, there are many factors that can damage these tissues.

Hair, for example, may be damaged by chemicals in care and treatment products, UV light, heat, and mechanical stress from styling. Genetic factors also affect hair density. Moreover, hair density frequently decreases with age and may be affected by other factors such as stress.

Likewise, skin may be damaged by UV light, chemicals, the drying effects of wind, and certain infectious agents, including certain bacteria, viruses, and fungi. The health and appearance of skin also typically decrease with age.

It is therefore readily apparent that there is a great need for effective compositions to improve the health of human hair, skin, and nails.

### DETAILED DESCRIPTION

The present disclosure provides compositions to deliver an active ingredient, silibinin, to human hair, skin, and/or nails. Silibinin (or silybin) is isolated from the milk weed (genus Silybum). This active ingredient is reported to have certain health-promoting benefits, including in vitro anticancer effects and liver-protective effects against certain toxins.

In certain embodiments, the disclosure provides compositions to deliver the active ingredient to the root bulb of the hair follicle. Silibinin is not soluble in water. Creating a cream formulation could possibly lead to clogging of pores at the site of the hair follicle, which could negatively affect restoration of the follicle. In certain aspects, the present disclosure relates to delivery of silibinin using oil-based formulations. Oil is absorbed into the stratum corneum and deeper layers of the skin where the root bulb cells are located.

Silibinin is soluble in acetone. We evaluated the possibility that silibinin could be first dissolved in acetone and then put into solution with an oil carrier. Since acetone evaporates to completion out of any open system, it could be removed leaving the silibinin in solution. One should keep in mind that acetone is incredibly flammable, and methods for removing acetone should be chosen carefully.

Certain aspects of the disclosure relate to compositions for stimulation of human hair growth. In certain embodiments, the composition comprises an effective amount of silibinin and an oil carrier. In some embodiments, the oil carrier comprises capric/capryllic triglycerides. Capric/capryllic acid triglycerides may be derived, for example, from glycerine and coconut oil or palm kernel oil.

In certain embodiments, the silibinin is present in the final composition at about 1%. In certain other embodiments, the silibinin is present in the final composition at less than 1%, between about 0.1% and 1%, about 0.1%, or less than 0.1%.

In certain embodiments, the composition may further comprise an effective amount of cycloastragalus as described in PCT/US14/38245, which is incorporated herein by reference. In some of these embodiments, the cycloastragalus is present in the final composition at about 1%, less than 1%, between about 0.1% and 1%, about 0.1%, or less than 0.1%

In certain embodiments, the composition may comprise an essential oil. Many essential oils have a long history of medical use. The composition may include an essential oil such as rosemary, thyme, cedarwood, chamomile, lavender, and/or geranium. Other essential oils may also be used.

In certain embodiments, the composition may comprise herbal extracts including, but not limited to, saw palmetto extract.

In certain embodiments, the composition may include other ingredients including, but not limited to, one or more salts to balance tonicity, stabilizers, cleansers, thickening agents, and fragrances.

Certain aspects of the disclosure relate to methods of preparing compositions for administering silibinin to the root bulb of the hair follicle. The method may include the steps of dissolving a desired amount of silibinin into acetone, adding capric/capryllic triglyceride, and removing the acetone. In certain embodiments, one or more essential oils, herbal extracts, or other ingredients may be added. In some embodiments, active oxygen species, such as ozone, may be used to thicken the composition to improve application.

Certain aspects of the disclosure relate to methods of improving hair density by administering a therapeutically effective amount of a composition comprising silibinin to the scalp of a patient in need thereof. In certain embodiments, the administration may be on a periodic basis, such as daily or weekly.

Certain other aspects of the disclosure relate to methods of stimulating human hair growth using compositions comprising silibinin.

The present disclosure also provides methods and compositions for improving the health and appearance of hair, skin, nails, and eyebrows. In certain embodiments, the disclosure provides a method for improving the health of at least one of hair, skin, nails, or eyebrows, comprising administering a composition which comprises an effective amount of silibinin and capric/capryllic triglycerides. In certain other embodiments, the present disclosure provides a composition for improving the health of at least one of hair, skin, nails, or eyebrows, wherein said composition comprises an effective amount of silibinin and capric/capryllic triglycerides. The composition may include any of the characteristics previously described.

Compositions for skin and/or nail care may be formulated as oil-based products, or they may be delivered as ointments, creams, or gels. Such compositions may be administered on a periodic basis, such as daily or weekly

To further illustrate these embodiments, the following examples are provided. These examples are not intended to limit the scope of the claimed invention, which should be determined solely on the basis of the attached claims.

### Example 1

A composition comprising silibinin was prepared using a process similar to that described in PCT/US14/38245 for the preparation of cycloastragalus compositions, although the preparation of a stable dispersion/solution of silibinin required significantly more time on the high shear mixer, which was unexpected. Briefly, a total desired amount of silibinin was dissolved into acetone at moderate temperature (40-50° C.). Capric/capryllic triglyceride was then added with mixing until completely mixed. The heat was then gently increased to evaporate the acetone completely (up to 60° C.). The solution was cooled with stirring. Rosemary EO 1 was added once the temperature was below 50° C., and the product was poured into dispensing containers for use by test subjects.

### Example 2

Compositions comprising other silibinin percentages (e.g., 0.1%) can be prepared using the methods recited in the previous examples.

### Example 3

Compositions for improving the health and appearance of hair, skin, nails, and eyebrows can be prepared using the methods recited in the previous examples.

### Example 4

Active oxygen species such as ozone may be injected into a composition by sparging with agitation. High-shear agitation may be used to create micelles.

It will be apparent to those having skill in the art that many changes may be made to the details of the above-described embodiments without departing from the underlying principles of the invention. The scope of the present invention should, therefore, be determined only by the following claims.

1. A composition for administering silibinin to the root bulb of the hair follicle, said composition comprising:
an effective amount of silibinin, and
an oil carrier.

2. The composition of claim 1, wherein the oil carrier comprises capric/capryllic triglycerides.

3-7. (canceled)

8. The composition of claim 1, further comprising an essential oil.

9. The composition of claim 8, wherein the essential oil is rosemary.

10. The composition of claim 1, further comprising an herbal extract.

11. The composition of claim 10, wherein the herbal extract is saw palmetto extract.

12. The composition of claim 1, further comprising a thickening agent.

13. The composition of claim 1, further comprising one or more ingredients selected from the group consisting of salts, stabilizers, cleansers, thickening agents, and fragrances.

14. A composition for administering silibinin to at least one of skin and nails, said composition comprising:
an effective amount of silibinin, and
an oil carrier.

15. The composition of claim 14, wherein the oil carrier comprises capric/capryllic triglycerides.

16-20. (canceled)

21. The composition of claim 14, further comprising an essential oil.

22. The composition of claim 21, wherein the essential oil is rosemary.

23. The composition of claim 14, further comprising an herbal extract.

24. The composition of claim 23, wherein the herbal extract is saw palmetto extract.

25. The composition of claim 14, further comprising a thickening agent.

26. The composition of claim 14, further comprising one or more ingredients selected from the group consisting of salts, stabilizers, cleansers, thickening agents, and fragrances.

27-31. (canceled)

32. A method for preparing a composition for administering silibinin to the root bulb of the hair follicle, said method comprising the steps of dissolving a desired amount of silibinin into acetone, adding capric/capryllic triglyceride, and removing the acetone.

33. The method of claim 32, wherein the silibinin is present in a concentration between 0.1% and 1%.

34. The method of claim 32, further comprising the step of adding an essential oil.

35. The method of claim 32, further comprising the step of adding an herbal extract.

36-48. (canceled)
